(19) United States
(12) Patent Application Publication
(10) Pub. No.: US 2024/0090829 A1
(43) Pub. Date: Mar. 21, 2024

(54) Evaluation of sleep data

(71) Applicant: Belun Technology (IP) Company Limited, Hong Kong, Hong Kong (CN)

(72) Inventors: Wenbo GU, Hong Kong (HK); Wenjie TSENG, Hsinchu City (TW)

(73) Assignees: Belun Technology (IP) Company Limited, Hong Kong (HK); Belun Technology (IP) Company Limited, Hong Kong (HK)

(21) Appl. No.: 17/642,698

(22) PCT Filed: Feb. 28, 2022
(86) PCT No.: PCT/CN2022/078160

§ 371 Date: Sep. 9, 2022

Related U.S. Application Data
(60) Provisional application No. 63/154,829, filed on Mar. 1, 2021.

(51) Int. Cl. A61B 5/00; A61B 5/024; A61B 5/1455

(52) U.S. Cl. CPC A61B 5/4815; A61B 5/02416; A61B 5/14551; A61B 5/4812; A61B 5/4818; A61B 5/7221; A61B 5/7267; A61B 5/7282; A61B 2562/0219

(57) ABSTRACT

The present technology relates to systems and methods for evaluating a user's sleep data. More particularly, the present technology contemplates a computer-implemented system or method of evaluating a quality of sleep for a user based on sleep data, such as by determining a set of quality metrics for the user's sleep based on the sleep data using a set of probabilistic models.

### FIELD OF THE INVENTION

The present disclosure generally relates to the field of computer-implemented systems and methods for measuring and/or determining physiological parameters of a user. More particularly, the disclosure relates to systems and methods for evaluating a user's sleep data.

### BACKGROUND OF THE INVENTION

Sleep is an important part of a person's overall health and well-being. Both the quantity of sleep as well as its quality may be correlated to the person's short-term and long-term health, as well as their overall well-being and quality of life.

Accordingly, it can be beneficial to obtain an improved understanding of a person's sleep habits and patterns, as well as its quality. A person's sleep habits and patterns may be quantified as the times and lengths of their sleep such as throughout a week or even over an extended period of time. A person's sleep quality may be also quantified by a number of measures, such as lengths of time in various stages of sleep, a number of arousal events, or occurrences of respiratory disorders, which may be for instance measured a number of apneas or hypopneas experienced throughout a night.

Although a number of methods and systems exist for quantifying a person's sleep quality and/or quantity, they may suffer from shortcomings such as high cost, complexity, inconvenience, noncompliance, or inaccuracy. As a result, there exists a need for improved systems and/or methods in this field for assessing a person's sleep.

### SUMMARY OF THE INVENTION

Even though sleep forms a significant part of a person's overall health and well-being, quantification of a person's sleep quality has been difficult and/or inaccessible for various reasons. One such reason is limitations or challenges in gaining access to hardware required to obtain the person's data, and another the difficulty in processing and evaluating the data once obtained. Although existing methods may require that the data be interpreted with human expertise, such access to experts may be expensive and/or inaccessible or many.

Difficulties may exist in tasks such as collection of data, processing and evaluation of data, and derivation of quality metrics as well as communication of the resulting quality metrics to the user. These difficulties may originate from challenges in access to expertise such as medical professionals whether they be doctors or sleep clinicians who may be able to evaluate the sleep data and produce the desired sleep quality metrics. Not only might such expertise be costly, but experts' time may also be limited as they are generally limited in supply. This situation may render these experts inaccessible for the purposes of longer-term sleep quality and health monitoring which may be perceived as less urgent and more time-consuming. In certain regions or to people of socio-economic backgrounds, such challenges may be even exacerbated.

This may be especially true for those users who desire, or require, health monitoring over an extended period of time. For at least these reasons, there exists a need or desire for an ability to determine and/or monitor a person's sleep quality consistently over time.

Still, such longer-term monitoring of sleep quality and health is very important for many in maintaining one's health and detecting any longer-term changes to it. For example, sleep disorders such as sleep apneas, and more specifically obstructive sleep apneas (OSAs) can be associated with significant morbidity and mortality of cardiovascular and pulmonary diseases.

The present disclosure thus contemplates a computer-implemented system or method of evaluating a quality of sleep for a user based on sleep data, such as by determining a set of quality metrics for the user's sleep based on the sleep data.

Advantageously, aspects of the present technology may assist in producing the set of sleep quality metrics in a speedy, consistent, low-cost manner that may help the to monitor the user's sleep health over time as well as any changes thereto.

In some forms, the present technology may allow long-term, high-quality monitoring of the user's health or sleep habits to identify any worsening of sleep quality such that preventative measures may be considered. For example, it may allow identification of sleep disorders that may indicate that the user may benefit from usage of medical devices such as positive airway pressure (PAP) devices. Aspects of the present technology may also allow the user to identify any impact of external factors on their sleep quality. It may also enable the user to monitor their own sleep health or general health, or for physicians to gain access to health data for their patient that they otherwise may not have.

Aspects of the present disclosure may relate to obtaining, processing and/or evaluating sleep data of a user in order to determine, quantify, and/or communicate a set of quality metrics for the user's sleep. The quality metrics may include, but not be limited to, values such as an AHI, a total sleep time, time in each of a plurality of sleep stages, a number of sleep events, or numbers of apneas and hypopneas. In some forms, the set of quality metrics may include a plurality of outputs, such as obtained for each of a predetermined period of time over the course of a sleep period.

In some cases, the quality metrics determined may be those known by those skilled in the art, such as an AHI, or amounts of time spent in each sleep stage (N1, N2, N3 or REM) during sleep.

In one form of the present disclosure, the sleep data may be received from a set of sensors worn by the user over the course of overnight sleep. The set of sensors may be integrally incorporated in one device, although they may be separate such as those that may be seen in a sleep laboratory for polysomnography (PSG). The sleep data may comprise a set of signals, such as one or more of a photoplethysmography (PPG) signal, an accelerometer signal, and an oxygen saturation (SpO2) signal. The set of signals may pre-processed, which may include removing low-quality signal portions from the set of signals. Additionally, or alternatively, the pre-processing may also include extracting another signal such as a pulse rate from the PPG or SpO2 signal, or it may include data transformation, such as transforming a signal from a time domain to frequency domain. The pre-processed data may be used as inputs to a prediction model to estimate the person's sleep state, and/or whether the person is undergoing a sleep event such as an apnea or a hypopnea. In one form, the pre-processed data may be used by a computer-implemented system incorporating a set of probabilistic prediction models to determine the times during which the user was sleep and a number of sleep events that occurred in the user's sleep duration.

In one form, a prediction model may include a set of neural networks, wherein each neural network is configured to receive the pre-processed data and probabilistically estimate the user's sleep state or a sleep event. Each neural network may be trained using a training dataset comprising pre-processed data as well as a set of labels indicating the target values of a variable the neural network is to predict. The neural network may be thus trained to optimise its performance in predicting the value of the variable for data which it has previously not been exposed to.

The set of prediction models may include a sleep state prediction model and/or a sleep event prediction model. A sleep state prediction model may be configured to from its inputs estimate the user's sleep state, such as by outputting a binary sleep state as asleep or awake. The sleep state prediction model may be further configured to estimate the user's sleep stage as awake, REM, N1, N2 or N3 throughout the pre-processed data. A sleep event prediction model may be configured to receive the pre-processed data and estimate a number of sleep events occurring in the data. The sleep event prediction model may be further configured to estimate whether each sleep event is an apnea or a hypopnea, and/or to estimate the time of occurrence for each sleep event.

Outputs from the set of prediction models may be used to determine a metric for sleep quality, such as an apnea-hypopnea-index (AHI) value. Additionally, or alternatively, the outputs from the set of neural networks may be used to generate a report detailing the user's sleep from the sleep data as determined by the set of neural networks. Accordingly, a system or a method according to one aspect of the present technology may estimate a quality of sleep for a user based on sleep data of the user.

One aspect of the present technology relates to a method for determining a sleep quality metric for a user, the method comprising the following operations performed via one or more processors: receiving a set of signals comprising a PPG signal, an SpO2 signal and an accelerometer signal, dividing the set of signals into segments, each segment comprising an equal number of pulses, processing each segment to determine a quality metric for each segment, dividing the set of signals into a first set of input segments, each input segment comprising a plurality of segments, selecting a second set of input segments from the first set of input segments based on the quality metrics for segments in each input segment, and determining the sleep quality metric for the user by operating on the second set of input segments using a first prediction model and a second prediction model, wherein the first prediction model determines whether the user is asleep from a first input, and the second prediction model determines occurrences of an apnea event or a hypopnea event using a second input, wherein the first input and the second input are based on the second set of input segments.

In one form, the first input further comprises a pulse rate.

In one form, each segment comprises one pulse.

In one form, each input segment spans between two and fifteen minutes, such as between four and six minutes.

In one form, the method further comprises determining the quality metric as acceptable or unacceptable.

In one form, the quality metric is determined by comparing the segment with a second segment.

In one form, the second segment comprises a pulse occurring before the segment.

One form of the method further comprises selecting the second set of input segments from the first set of input segments by selecting each input segment that comprises fewer than 40% of segments marked as unacceptable.

In one form, the sleep quality metric is an AHI value.

In one form, the first prediction model and the second prediction model are neural networks.

In one form, the first prediction model and the second prediction model comprise the same neural network structure.

One form of the method further comprises determining an amount of time the user was asleep in the second set of input segments.

One form of the method further comprises determining a number of apnea or hypopnea events present in each input segment in the second set of input segments.

One form of the method further comprises further comprising the first prediction model determining a sleep stage to one of: awake, NREM or REM states.

In one form, the first prediction model determines the sleep stage to each 30-second output segment.

One form of the method further comprises the second sleep prediction model outputting a number of apnea events and a number of hypopnea events in each of the second set of input segments.

One aspect of the present technology relates to a system for determining the sleep quality metric for a user, comprising: a memory storing instructions; one or more processors configured to execute the instructions to perform one or more operations, the operations comprising the method; and the set of sensors configured to generate the PPG signal, the SpO2 signal and the accelerometer signal from the user's sleep session.

### BRIEF DESCRIPTION OF THE DRAWINGS

FIG. 1 shows an example schematic of a system for determining a metric for sleep quality, according to one form of the present technology.

FIG. 2 shows an example flowchart for determining a metric for sleep quality, according to one form of the present technology.

FIG. 3 shows an example flowchart for evaluating a pulse waveform quality according to one form of the present technology.

FIG. 4 shows an example structure of a suitable prediction model according to one form of the present technology.

### DETAILED DESCRIPTION

One aspect of the present technology relates to methods and systems for probabilistically evaluating a set of sleep quality metrics such as an AHI.

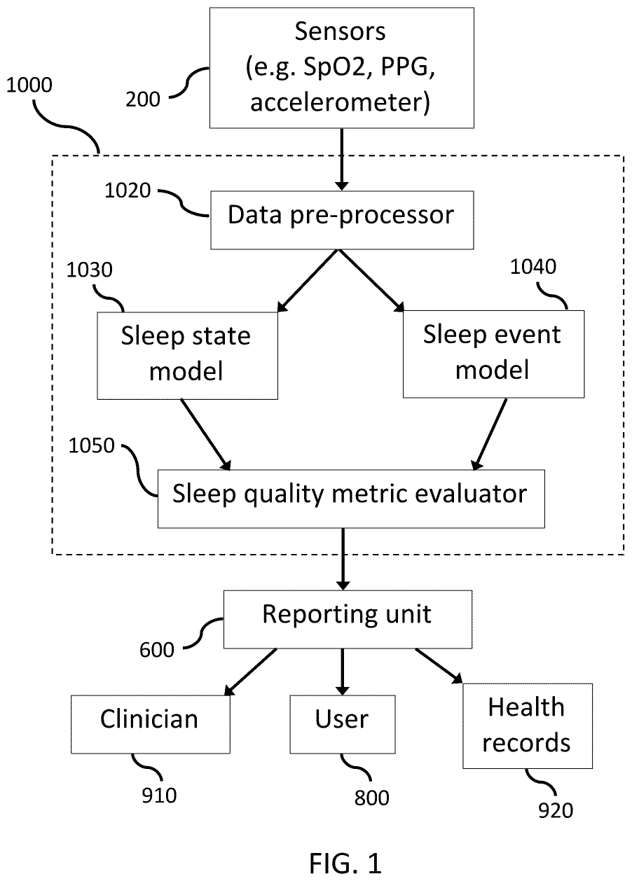

FIG. 1 shows a schematic of a system 1000 for determining a user's sleep quality metric according to one form of the present technology. The system 1000 comprises a data pre-processor 1020, a sleep state prediction model 1030, a sleep event prediction model 1040 and a sleep quality metric evaluator 1050.

In some arrangements of the present technology, data for a user's sleep session (i.e. sleep data) may be acquired via a set of sensors 200, which may or may not form a part of the system 1000. The data pre-processor 1020 may be configured to receive the sleep data from the set of sensors 200, such as by being in electronic communication, whether directly or otherwise such as through a network. The pre-processor 1020 may be connected to one or more prediction models such as a sleep state prediction model 1030 and/or a sleep event prediction model 1040, wherein each prediction model is configured to determine one or more outputs based on inputs from the pre-processor 1020. Outputs from the prediction models 1030 and 1040 may be communicated to the sleep quality metric evaluator 1050 in order to determine one or more metrics indicative of quality of the user's sleep, such as an AHI value. The determined value may be delivered to a reporting unit 600, before being further processed such as a part of a display to be shown or report to be sent to a user 800, or to a clinician 910 or health records management system 920.

The system 1000 may comprise a computing device, such as a portable or wearable device comprising software and/or hardware for implementing one or more aspects of the present technology. The system 1000 may comprise multiple, connected computing devices, such as through a local network or the Internet. In fact, the system 1000 may comprise a communication network such as a local area network (LAN), cellular network, a near field communication (NFC) connection, a wired network or any protocols that may allow communication therethrough. Thus, one or more components of the system 1000 may be connected with each other through one or more networks or a part thereof.

A computing device may comprise a processor and a memory of non-transitory computer-readable medium for storing computer programs or code to be executed by the processor. The processor may comprise one of any number of available devices for executing instructions or code, such as a digital or analogue processor. The memory may comprise one of any number of devices capable of electronically storing information, such as optically readable media, magnetically readable media, electrical charge-based storage media or solid-state storage media.

A method or process according to the present technology, or a part thereof, may be implemented on a computing device. The memory may store instructions for executing at least some of the operations for performing the method or process, which may be executed by the processor.

In one form, a system 1000 may comprise one computing device configured to perform one or more of the methods and processes described in the present disclosure. In other forms, a computing system may comprise a plurality of computing devices, wherein each computing device is connected to each other through a communication network, and together perform one or more of the methods and processes described in the present disclosure. Accordingly, the present disclosure contemplates a tangible, non-transitory computer-readable medium storing instructions for execution by a processor to estimate a quality of sleep for a user based on sleep data of the user.

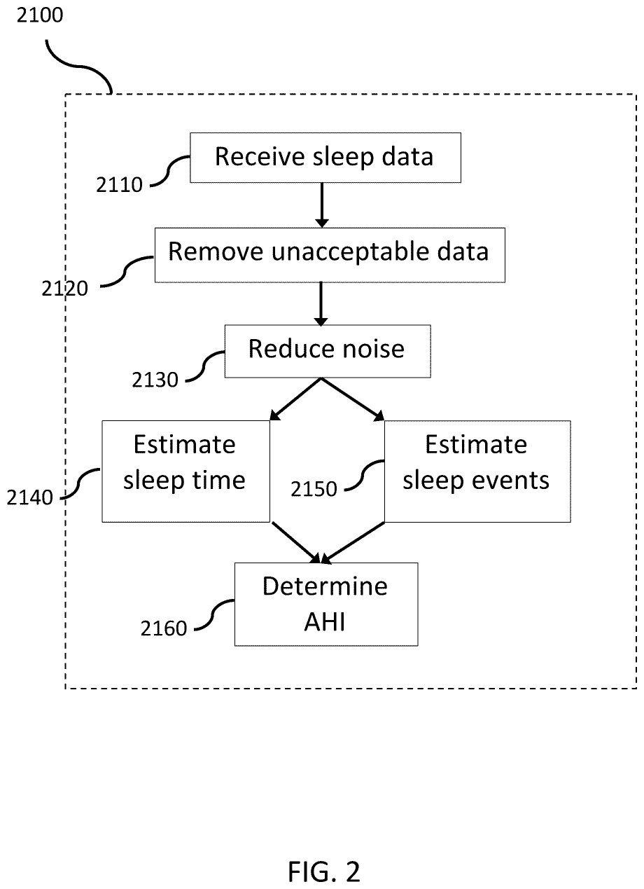

An example process 2100 to determine a set of sleep quality metrics is shown in FIG. 2. The process begins at step 2110 by receiving sleep data, such as from the set of sensors 200. At step 2120, the sleep data may be processed to remove unacceptable data, and further processed at step 2130 to reduce noise. The processed sleep data may be used to probabilistically estimate a total sleep time at step 2140 and probabilistically estimate a number of sleep events at step 2150. The resulting outputs may be used to determine an AHI value at step 2160.

Computer-implementation of processes such as the above may advantageously allow accurate, cost-effective and consistent monitoring of a user's sleep quality, especially for the medium to longer term monitoring.

The process 2100 may be performed at the completion of a sleep session, whereupon the data from the entire sleep session may be evaluated. Additionally, or alternatively, the process 2100 may be performed as the input sleep data is being received, such as during an overnight sleep session. The process 2100 may be performed as the user sleeps, evaluating for example as the data is generated, or the process 2100 may be performed at a regular and/or predetermined interval, such as every few minutes, or hours.

The process 2100 begins with receiving sleep data at step 2110, such as from the sensors 200 (as shown in FIG. 1). In one form, the sensors 200 may be included in a wearable health monitoring device, such as one disclosed in United States Patent Application publication number US2018/0132789A1. In other arrangements, the sleep data may be acquired from a plurality of devices, such as those used in polysomnography (PSG) measurements. The sleep data may additionally or alternatively be acquired from any number of sources, such as from a remote database via a network connection.

The sleep data may comprise a set of signals indicative of the sleep session, for example the set of signals having been recorded over at least a portion of the sleep session. The set of signals may comprise one or more of a photoplethysmography (PPG) signal, an accelerometer signal, an oxygen saturation (SpO2) signal and a pulse rate signal. One or more of the set of signals may be measured directly by a sensor, such as a PPG sensor measuring a PPG signal, or an SpO2 sensor measuring an SpO2 signal. Additionally, or alternatively, one or more signals, such as a pulse rate, may be inferred from another signal such as a PPG signal or an SpO2 signal.

A signal may be a time-domain signal recorded at a regular interval or rate during the sleep session, such as at 0.5 Hz, 1 Hz, 5 Hz, 20 Hz, 50 Hz, or 100 Hz. In some forms, each signal may be recorded at the same rate such as 5 Hz, 20 Hz or 50 Hz. In other forms signals may be recorded at different rates to each other, such as one at 1 Hz and another at 20 Hz. In one example, the SpO2, pulse rate and accelerometer signals may be sampled at 1 Hz, 2 Hz or 5 Hz, while the PPG signal may be recorded at 20 Hz or 50 Hz. The recording rate for each signal may be predetermined.

The sleep data may be pre-processed before being used as input data for one or more prediction models. The sleep data may be pre-processed for one or more reasons such as: segmentation, data quality, noise reduction, normalisation, or domain transformation. In one form, a process 2100 may comprise two pre-processing steps 2120 and 2130 as shown in FIG. 2. The pre-processing may improve the accuracy of the prediction model, or improve the quality of data, such as by removing data segments from when the user has yet to go to sleep or when the data is of low quality. Each signal in the set of signals may undergo the same set of pre-processing steps, or some signals may undergo different sets of pre-processing steps to others.

The data pre-processor 1020 as shown in FIG. 1 may carry out pre-processing operations. The data pre-processor 1020 may comprise one or more sub-modules, such as to perform one or more of its pre-processing operations such as data quality evaluation, de-noising, segmentation and transformation. The data pre-processor 1020 may receive a set of signals such as SpO2, PPG, motion and pulse rate signals and perform one or more pre-processing operations on one or more of the set of signals. The pre-processor 1020 may perform the same pre-processing operations to each of the set of signals, or perform different pre-processing operations to at least some of the set of signals.

In one form, the data pre-processor 1020 may sequentially perform a predetermined set of pre-processing operations, such as quality evaluation, filtering based on evaluated quality, de-noising, segmentation and transformation, at the completion of which it may deliver a first output to the sleep stage prediction model 1030 and a second output to the sleep event prediction model 1040.

The data pre-processor 1020 may perform data quality evaluation by dividing the set of received signals into segments and determining a data quality of each segment. Each segment may be evaluated based on a predetermined set of criteria, such as to be marked as “acceptable” or “unacceptable”. Each segment may comprise a number of pulses, such as one, two, five or ten. In one form, the pre-processor 1020 may assess acceptability of each segment based on its pulse waveform, user motion data and/or arterial pulse intensity. By doing so and reducing or removing from the input dataset spurious data such as low-quality data or non-sleep data, the resulting overall evaluation of sleep quality or sleep quality metric may be improved.

A segment data quality evaluation by its pulse waveform may be carried out based on a dynamic time-warping (DTW) difference between the segment and a previous segment, as well as whether the previous segment was marked as acceptable.

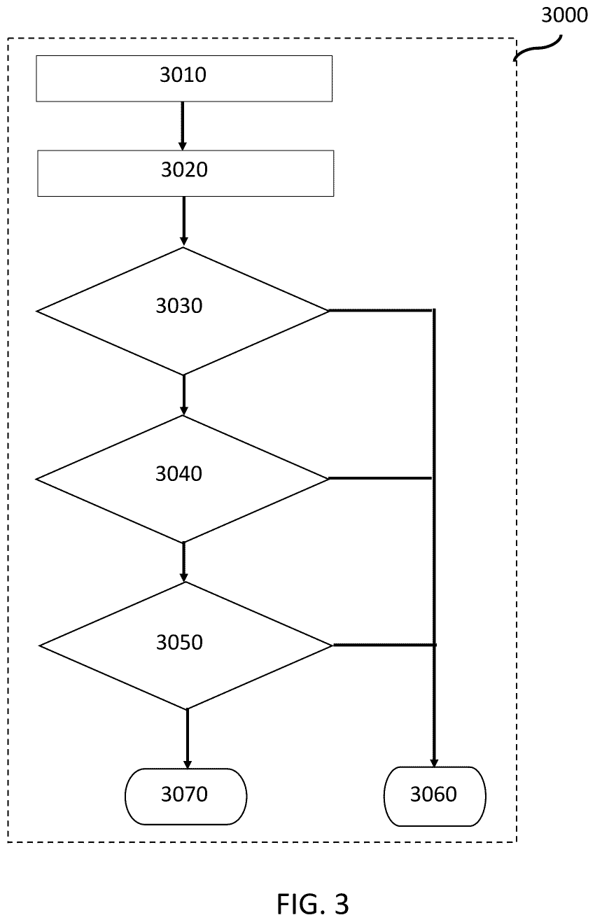

An example pulse waveform quality evaluation process 3000 is shown as a flowchart in FIG. 3. In step 3010, a processor performing the quality evaluation may receive a data segment and determine a DTW distance with a previous segment in step 3020. At step 3030, the processor may determine whether the previous segment was also marked unacceptable and the DTW distance is below a first threshold, and if so the segment will be marked unacceptable at step 3060. If not, the processor will determine at step 3040 whether the DTW difference is above a second threshold and the previous segment was marked acceptable, where if so the segment will be marked unacceptable at step 3060. If not, at step 3050, the processor will determine if the DTW is above a third threshold, and if so the segment may be marked unacceptable at step 3060. If not, the segment will be marked acceptable at step 3070.

A data segment may also be evaluated based on the motion data for the data segment, such as to reduce from the dataset any segments during which the user may have been awake. To evaluate a segment data quality by the user motion data, the segment's movement data may be compared against an overall RMS threshold value, such as a predetermined value indicative of the user being asleep. The movement data may have been measured by an accelerometer in the set of sensors 200. For a segment with a pulse at time t, its measured movement in the x, y and z directions may be compared with the previous pulse at time t−1, a root square difference between the two segments \sqrt{(xₜ−xₜ₋₁)2+(yₜ−yₜ₋₁)2+(zₜ−zₜ₋₁)2} may be compared with a threshold to determine its acceptability.

A segment data quality may also be evaluated by its arterial pulse intensity, such as to reduce data segments of potentially low quality. The arterial pulse intensity may be derived as the difference between the peak-to-trough value of the pulse, compared with a threshold value.

A segment data may be evaluated to be of acceptable quality if all of the predetermined criteria are met. For example, a segment data evaluation algorithm may evaluate a segment's user motion data, arterial pulse intensity, and pulse waveform quality in that order, marking unacceptable any data segment that fails any of the criteria. In some forms of the present technology, a segment data may be evaluated to be of acceptable quality if a minimum percentage of predetermined criteria (e.g. 2 of 3) are met.

Upon completion of the data quality evaluation process, the quality evaluation outputs may be used to determine how much of the sleep data to accept and/or to reject. In one form, the sleep data may be divided into blocks, wherein each block comprises one or more segments with data quality evaluation markers. Each block may then be either accepted or rejected based on the ratio of segments contained therein. For example, a block may be between 2 and 15 minutes long, such as between 3 and 10 minutes, such as 5 minutes. The block may be marked for rejection if it contains more than over 15% of unacceptable segments, such as 25%, 35%, or 50%.

The accepted blocks of sleep data may be subsequently filtered for de-noising to further improve its signal quality. A Hampel filter or an averaging smoothing filter may be examples of such suitable filters, although any number of other filters, or neural network methods, may be also suitable. Thus the filters may output filtered blocks of sleep data. Additionally, or alternatively, the pre-processor 1020 may perform transformations of sleep data, such as after the quality evaluation and/or de-noising.

A number available transformations may be suitable for application to the data at the point of collection, or subsequently (e.g. after quality evaluation) to transform the data prior to further processing or use as inputs. Examples of applicable transformations may include generating an FFT spectrogram, a Markov Transition Field, a recurrence plot of a Gramian Angular Field. Such transformed data may form inputs in addition to, or as alternatives to, any of the aforementioned data blocks. In one form, the pre-processor may transform the data to produce a signal that is appended to the sleep data. Thus, an output from the pre-processor may include a set of data comprising the input signal channels as received by the pre-processor well as any additional channels produced as a result of data transformations.

Accordingly, the pre-processor 1020 may receive input sleep data and output processed sleep data that may have been filtered to include less of low-quality or undesirable data, filtered to improve its quality and transformed.

The pre-processed data may form inputs to one or more prediction models configured to evaluate the pre-processed data. The one or more prediction models may include a sleep state prediction model 1030 for estimating whether the user was asleep or awake, and a sleep event prediction model 1040 for estimating whether an event such as an apnea or hypopnea as shown in FIG. 1.

In one form, the sleep state prediction model may output a length of awake time in the pre-processed data as shown in step 2140 of FIG. 2, or a length of asleep time in the pre-processed data, and the sleep event prediction model may output a number of total sleep events detected in the pre-processed data as shown in step 2150 of FIG. 2.

A prediction model may assume any of a number of available forms, such as a set of algorithms, a statistical prediction model, a machine learning model, a neural network or a combination of model types. A neural network may comprise a set of artificial neurons, or units, which are connected to other units of the neural network to process signals sent therethrough, together forming a network which may produce a set of outputs from a set of inputs. A neural network may comprise a set of layers where each layer may comprise a set of artificial neurons. In some forms a signal may traverse forward through layers to process the data, while errors are propagated backwards while building the model, such as adjusting parameters of each individual neurons and/or its connections. In other forms, signals within a neural network may be less linearly organised.

A prediction model such as a neural network may be built using a training dataset, wherein outputs of the target variables to be estimated by the prediction model are included. This is also referred to as a training dataset in machine learning. A machine-learned prediction model such as a neural network may be self-trained, which is to say that its parameters may be at least partly determined based on a training dataset in order to optimise for a predetermined metric such as a difference between the prediction and the known (target) output. Thus, a machine learning prediction model may be constructed without all aspects of its prediction mechanism being explicitly programmed, as some of its prediction mechanism may be learned.

For example, the training dataset may comprise a set sleep data of same type as input data to be used in a system such as system 1000 of FIG. 1 or process 2100 of FIG. 2. More specifically, the training data may comprise the same set of signals or channels as the input data to be used in the system 1000. Additionally, the training dataset may comprise target values of output variables such as the sleep state and occurrence of any sleep events, as would be output in steps 2140 and 2150 respectively in FIG. 2. A training process for a sleep state prediction model may thus comprise setting a target variable of sleep state, and automatically iterating, based on the measured differences between target sleep state such as the measured sleep state and the predicted sleep state as produced by the model. Over the course of the training process the prediction model's performance would improve as its parameters (i.e. weightings) are refined through processes such as backpropagation.

A training process may comprise, or be preceded by, collection of training data from a set of users, such as 50, 100, or 200 users, to build a training dataset. The training data may include the same set of input data signals, or channels, as would be used in the prediction model, such as one or more of SpO2, PPG, movement, pulse rate data. Additionally, the training data may include a sleep state signal and a sleep event signal to be used as the target variable. The sleep state signal and/or the sleep event signal may be collected or measured using an external system such as a polysomnography and an automated device, although they may also be determined by an expert reviewing the data manually such as in real-time while the user is asleep or subsequent to the user's sleep session.

For example, polysomnography signals may be able to determine with high accuracy whether a user is asleep, through one or more of an electroencephalogram (EEG), an electrooculogram (EOG) and an electromyogram (EMG). Thus, the sleep state may be determined using a computer-implemented system based on the EEG, EOG and/or the EMG data. Polysomnography signals may also be able to determine with high accuracy whether a user is undergoing an apnea or hypopnea event by monitoring the user's oxygen saturation signal and chest effort signal as well as the sleep state. Thus, such values can be used as the target, or truth, values which the prediction model is to aim to produce given a set of input data.

A prediction model may use as inputs a set of sleep data, such that it might estimate the user's length of sleep or number of events. The sleep data may be in one or more forms as previously discussed, such as the filtered blocks of sleep data or the accepted blocks of sleep data.

In one form, the set of prediction models in a system or method according to the present technology may comprise two neural networks. A first neural network (sleep state prediction model) may be configured to probabilistically estimate whether the user was asleep (sleep state) given a set of data, and a second neural network (event detection prediction model) may be configured to probabilistically estimate whether the user was experiencing a sleep event such as an apnea or a hypopnea (event detection) given a set of data.

A prediction model's input dataset may comprise one or more of SpO2, PPG, accelerometer, pulse rate and FFT spectrography channels. In turn, the prediction model may generate an output indicative of a probability, such as whether the user was asleep or whether the user was experiencing a sleep event.

As those skilled in the art would understand, a number of neural network or machine learning architectures may be suitable in achieving this goal.

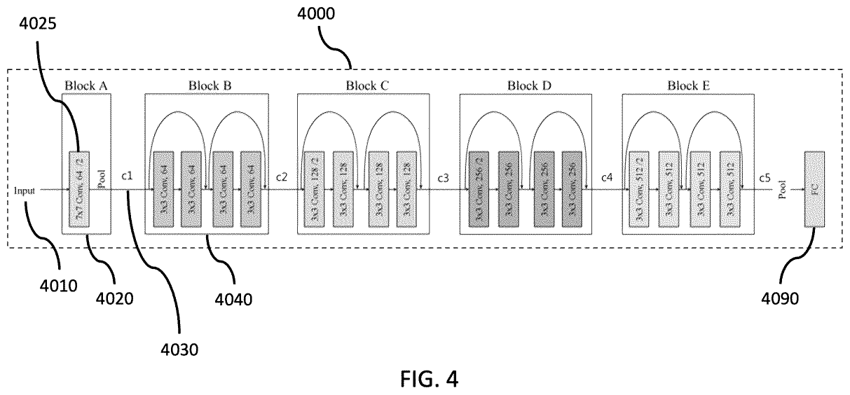

FIG. 4 shows one suitable example structure of a prediction model, wherein the prediction model is a neural network 4000. The first block of layers 4020 may receive and process an input 4010, starting from its first layer 4025. An output from the first block of layers 4020 may then be passed onto the second block of layers 4040 through a first connection 4030, and similarly through the subsequent blocks until it is delivered to an output layer 4090. The output layer 4090 may produce as its output a probabilistic prediction, such as whether the data indicates that the user is asleep, or whether the user is undergoing a sleep event. FIG. 4 shows each layer such as 4025 to be a convolutional layer, although other layer types may be suitable.

In one example, the sleep state prediction model may comprise a set of convolutional blocks, wherein each convolutional block comprises a set of convolutional layers. The sleep state prediction model may assume, or approximate, an architecture of a ResNeXt network comprising 25 layers. A convolutional layer may be fully-connected to the next, or be connected via a bottleneck, such as in order to discourage overfitting. In one form, a squeeze-and-excitation module, otherwise known as an SENet, may be added in each residual block. The sleep state prediction model's activation function may be ReLu, GELU or Swish.

In some forms, the sleep state prediction model may output a series of values indicating times that the user was asleep and times when the user was awake throughout the pre-processed data. In another form, the sleep state prediction model may output a sleep state, as well as a sleep stage if the user is asleep, such as one of REM, N1, N2 or N3 sleep.

The outputs from the one or more prediction models may be used to determine a set of quality metrics for the user's sleep. The set of quality metrics may be an AHI value, calculated as the number of total sleep events divided by sleep time (in hours). The resulting set of quality metrics may subsequently be stored, communicated to the user or transmitted for storage or further evaluation such as to a health record database.

Thus, as shown in FIG. 5, a system may comprise a set of prediction models comprising two neural networks. The first neural network is configured to probabilistically estimate a sleep state, and the second neural network is configured to probabilistically estimate occurrence of a sleep event. The first neural network, the sleep state network, may be configured to receive a sleep state input data comprising a set of features, such as a movement feature, a PPG feature and a pulse rate feature, and deliver an output indicative of whether the user is asleep. The second neural network, the sleep event network, may be configured to receive a sleep event input data comprising set of features, such as a SpO2 feature and a PPG feature, and deliver an output indicative of whether the user is experiencing a sleep event.

Each of the input set of features to a neural network may comprise filtered and/or a transformed data as described above. For example, a movement feature may include filtered movement data as output by the pre-processor to remove low quality segments and de-noised from a measured accelerometer signal. Additionally, the movement feature may include transformed data wherein the measured accelerometer signal has been transformed using to a frequency domain signal using FFT. In operation, a neural network may receive the input data, whereupon it would be propagated forward through the neural network's layers and processed to produce an output.

The sleep state network, for the given input data, produce a likelihood of the user's sleep state. For example, the sleep state network may output for each 5-minute segment of input data a binary state estimating whether the user was asleep or awake. In another example, the sleep state network may output for each 5-minute segment of input data one of a plurality of sleep states, such as one of awake, REM, N1, N2 or N3.

The sleep event network may, for the given input data, produce a likelihood of the user experiencing a sleep event. For example, the sleep event network may output for each 10-second segment of input data a binary state estimating whether the user was experiencing a sleep event. In another example, the sleep state network may output for each 5-minute segment of input data one of a total number of sleep events that the user may have experienced.

One aspect of the present technology relates to determining a set of quality metrics for the user's sleep. In one form, the set of quality metrics may be a sleep length metric to indicate the amount of time that the user was asleep for, and/or a sleep event metric to indicate the number of events that the user experienced during sleep. The set of quality metrics may be an AHI value. The set of quality metrics may be determined from the outputs from the probabilistic models, such as the set of neural networks comprising a sleep state network and a sleep event network.

In one form, the system 1000 may comprise a sleep quality metric evaluator configured to receive outputs from the probabilistic models and to determine a set of quality metrics.

The set of quality metrics may be delivered to one or more recipients, such as the user, their health care provider, and/or to a database. In one form, a reporting unit may prepare a visual alert to be displayed to the user on a screen, such as on a computing device. In another form, a reporting unit may prepare and transmit an e-mail to a user or a healthcare provider. In yet another form, a reporting unit may be in communication with a database, whereupon the reporting unit may populate the database with the set of quality metrics. The reporting unit may be configured to deliver the set of quality metrics at a predetermined interval, such as every day in the morning, or once a week at a predetermined time.

Thus, the user, healthcare provider or an enquirer to the database may conveniently monitor the user's sleep quality in a consistent manner, such as over a short term to assess a quality of sleep or over a longer term to assess any potential changes to the user's health.

It is to be understood that the foregoing disclosure is merely illustrative of the application of the principles of the invention. For instance, it is also to be understood that aspects of the present disclosure, such as a process, or a method, may be implemented with one or both of hardware and software instructions.

Reference herein to details of any illustrated examples or embodiments is not intended to limit the scope of the claims. Other examples or embodiments, such as based on combination of portions of the present disclosure, may be apparent from consideration of the present disclosure.

1. A method for determining a sleep quality metric for a user, the method comprising the following operations performed via one or more processors:
receiving a set of signals comprising a PPG signal, an SpO2 signal and an accelerometer signal;
dividing the set of signals into segments, each segment comprising an equal number of pulses;
processing each segment to determine a quality metric for each segment;
dividing the set of signals into a first set of input segments, each input segment comprising a plurality of segments;
selecting a second set of input segments from the first set of input segments based on the quality metrics for segments in each input segment, and
determining the sleep quality metric for the user using a first prediction model and a second prediction model, wherein the first prediction model determines whether the user is asleep from a first input, and the second prediction model determines occurrences of an apnea event or a hypopnea event using a second input, wherein the first input and the second input are based on the second set of input segments.

2. The method of claim 1, wherein the first input further comprises a pulse rate.

3. The method of claim 2, wherein each segment comprises one pulse.

4. The method of claim 3, wherein each input segment spans between two and fifteen minutes.

5. The method of claim 4, wherein each input segment spans between four and six minutes.

6. The method of claim 5, further comprising determining the quality metric as acceptable or unacceptable.

7. The method of claim 6, wherein the quality metric is determined by comparing the segment with a second segment.

8. The method of claim 7, wherein the second segment comprises a pulse occurring before the segment.

9. The method of claim 8, further comprising selecting the second set of input segments from the first set of input segments by selecting each input segment that comprises fewer than 40% of segments marked as unacceptable.

10. The method of claim 9, wherein the sleep quality metric is an AHI value.

11. The method of claim 10, wherein the first prediction model and the second prediction model are neural networks.

12. The method of claim 11, wherein the first prediction model and the second prediction model comprise the same neural network structure.

13. The method of claim 11, further comprising determining an amount of time the user was asleep in the second set of input segments.

14. The method of claim 11, further comprising determining a number of apnea or hypopnea events present in each input segment in the second set of input segments.

15. The method of claim 11, further comprising the first prediction model determining a sleep stage to one of: awake, NREM or REM states.

16. The method of claim 15, wherein the first prediction model determines the sleep stage to each 30-second output segment.

17. The method of claim 11, further comprising the second sleep prediction model outputting a number of apnea events and a number of hypopnea events in each of the second set of input segments.

18. A system for determining the sleep quality metric for a user, comprising:
a memory storing instructions;
one or more processors configured to execute the instructions to perform one or more operations, the operations comprising the method of claim 1; and
the set of sensors configured to generate the PPG signal, the SpO2 signal and the accelerometer signal from the user's sleep session.
